(19) United States
(12) Patent Application Publication
(10) Pub. No.: US 2024/0408001 A1
(43) Pub. Date: Dec. 12, 2024

(54) USE OF A BLUEBERRY EXTRACT TO IMPROVE THE AESTHETIC APPEARANCE OF SKIN

(71) Applicant: SOCIETE D'EXPLOITATION D PRODUITS POUR LES INDUSTRIES CHIMIQUE SEPPIC, Paris (FR)

(72) Inventors: Catherine KERN, CASTRES (FR); Rémi LAVILLE, PONTRIEUX (FR)

(21) Appl. No.: 18/689,706

(22) PCT Filed: Sep. 28, 2022
(86) PCT No.: PCT/EP2022/077067

§ 371 Date: Mar. 6, 2024

(30) Foreign Application Priority Data
Sep. 29, 2021 (FR) FR2110266

(51) Int. Cl. A61K 8/9789; A61K 8/02; A61K 8/11; A61K 8/49; A61K 31/352; A61K 36/45; A61Q 19/00

(52) U.S. Cl. CPC A61K 8/9789; A61K 8/0229; A61K 8/11; A61K 8/498; A61K 31/352; A61K 36/45; A61Q 19/00; A61K 2800/85; A61K 2800/92

(57) ABSTRACT

Use of a blueberry extract to improve the aesthetic appearance of human skin, the improvement of said aesthetic appearance including coloration and the homogeneity of the complexion, as well as a reduction in roughness.

The present invention relates to the use of an extract of blueberries for improving the esthetic appearance of human skin.

The skin constitutes the interface between the organism and the external environment. Its purpose is to ensure the protection of the human body by forming a true barrier which is vital.

The complexion of the skin is also a reflection of a state of well-being, often associated with youth, vitality, dynamism,

There has long been a trade in products tending to avoid a deterioration in the complexion of the skin.

This is because, whether they are of a physical (mechanical, thermal, UV radiation, and the like, factors), chemical (surfactants, solvents, allergens, and the like) or biological (infectious agents) nature, the skin is daily subjected to multiple attacks.

The term “normal skin” is widely used to describe balanced skin. Normal skin has good blood circulation, a soft, smooth and velvety texture, a fresh translucent appearance and a uniform pink color; it is free of imperfections, and is not sensitive.

When the cutaneous barrier is intact, it can act more effectively to retain water and protect against external attacks. Skin with a strong and healthy cutaneous barrier is more beautiful and functions better over time.

External factors, such as exposure to pollution, to UV radiation, to variations in temperature and/or in humidity, to tobacco smoke, or also, among the intrinsic factors, stress, fatigue, hormonal changes, aging or excessive sebum secretion, induce harmful effects on all the components of the barrier function of the skin and can in particular result in a deterioration in the complexion of the skin. More particularly, when exposed to these factors, the skin tends toward a nonuniform and whitish complexion.

The term “complexion of the skin” is understood to mean both the color of the skin and its uniformity.

In order to mask the unsightly manifestations of the deterioration in the complexion of the skin, makeup products, such as foundations, are applied to the skin but these often comprise ingredients of synthetic origin obtained from starting materials of fossil origin. This solution clashes with the expectations of consumers desiring an orientation toward cosmetic products of natural origin.

Starting from this, a problem which arises is that of developing a composition of plant origin which makes it possible to improve the esthetic appearance of human skin, the improvement in said esthetic appearance comprising both that in its coloration and in the uniformity of its complexion, and also the decrease in its roughness.

This is why a subject matter of the invention is the use of an extract of blueberries for improving the esthetic appearance of human skin, the improvement in said esthetic appearance of human skin comprising both that in its coloration, in the uniformity of its complexion and also the decrease in its roughness.

The term “coloration of the skin” is understood to mean, in the context of the present invention, to give the skin a “good look effect”, restoring to it coloration or a darker complexion, as explained in the experimental part. The term “coloration of the skin” used in this context does not mean the tanning of the skin induced by UV rays or a chemical product applied to the skin.

The term “uniformity of the complexion of the skin” is understood to mean, in the context of the present invention, a uniform color of the skin without irregular pigmentation or reduced pigmentation.

The term “decrease in the roughness of the skin” is understood to mean, in the context of the present invention, an improvement in the texture of the skin and thus a smooth skin grain.

The extract of blueberries employed in the use according to the invention can be either fermented or non-fermented.

According to a particular aspect of this use, the extract of blueberries employed is an extract of fermented blueberries.

Depending on the case, the use according to the invention can exhibit one or more of the following characteristics:
the extract of blueberries is provided in the form of a powder;
the extract of blueberries comprises, per 100% of its own weight, between 1% and 10% by weight of water and between 90% and 99% by weight of dry matter (DM), more particularly between 1% and 8% by weight of water and between 92% and 99% by weight of dry matter (DM) and more particularly still between 1% and 7% by weight of water and between 93% and 99% by weight of dry matter (DM);
the extract of blueberries comprises:
a) at least one compound of formula (I):

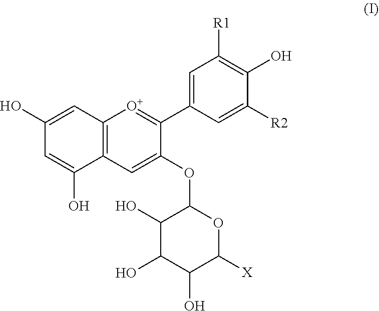

with R1 and R2 identical or different and representing a hydrogen atom —H, the hydroxyl radical —OH or the methoxyl radical —OCH₃,
and
with X═—H (L-arabinose) or —CH₂—OH (D-glucose, D-galactose), and
b) at least one compound of formula (II):

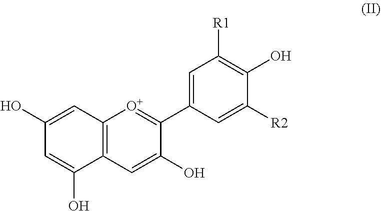

with R1 and R2 identical or different and representing a hydrogen atom —H, the hydroxyl radical —OH or the methoxyl radical —OCH₃;
the extract of fermented blueberries comprises:
c) at least one compound of formula (III):

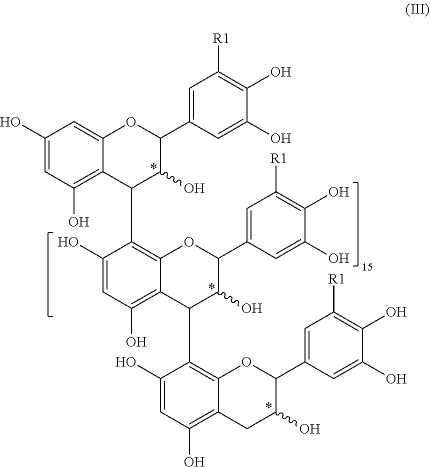

with, independently for each repeat and constituent unit of the compound of formula (III):
   R1 representing a hydrogen atom —H or the hydroxyl radical —OH, and
   it being possible for the asymmetric carbons indexed by asterisks * in the formula (III) to be of R or S configuration;
the extract of fermented blueberries comprises, for example, per 100% of its weight:
   from 20% to 50% by weight, more particularly from 25% to 50% by weight, more particularly still from 30% to 50% by weight, of at least one compound of formula (I),
   from 0.01% to 5% by weight, more particularly from 0.01% to 4% by weight, of at least one compound of formula (II),
   from 1% to 5% by weight, more particularly from 1% to 4% by weight, of at least one compound of formula (III),
   from 1% to 10% by weight, more particularly from 1% to 8% by weight, more particularly still from 1% to 7% by weight, of water,
   from 30% to 78.99% by weight, more particularly from 32% to 72.99% by weight, more particularly still from 35% to 67.99% by weight, of at least one compound chosen from the group consisting of carbohydrates, inorganic salts and proteins,
   it being understood that the sum of the various constituents of this extract of fermented blueberries is equal to 100%;
the extract of fermented blueberries additionally comprises at least one compound of formula (IV):

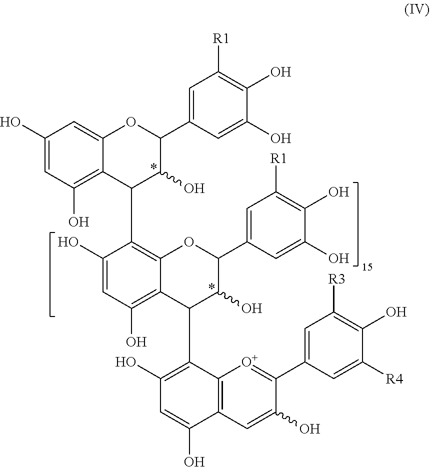

with, independently for each repeat and constituent unit of the compound of formula (IV):
   R1 representing a hydrogen atom —H or the hydroxyl radical —OH, and
   it being possible for the asymmetric carbons indexed by asterisks * in the formula (IV) to be of R or S configuration,
and
with R3 and R4 identical or different and representing a hydrogen atom —H, the hydroxyl radical —OH or the methoxyl radical —OCH₃;
the extract of fermented blueberries comprises, for example, per 100% of its weight:
   from 0.1% to 5% by weight, more particularly from 0.1% to 4% by weight, more particularly still from 0.1% to 3% by weight, of at least one compound of formula (I) (anthocyanin),
   from 0.01% to 1% by weight, more particularly from 0.01% to 0.8% by weight, of at least one compound of formula (II) (anthocyanidin),
   from 0.5% to 10% by weight, more particularly from 0.5% to 8% by weight, more particularly still from 0.5% to 6% by weight, of at least one compound of formula (III) (proanthocyanidin),
   from 10% to 50% by weight, more particularly from 10% to 40% by weight, more particularly still from 10% to 35% by weight, of at least one compound of formula (IV) (pseudoproanthocyanidin),
   from 1% to 10% by weight, more particularly from 1% to 8% by weight, more particularly still from 1% to 7% by weight, of water,
   from 24% to 88.39% by weight, more particularly from 39% to 88.39% by weight, more particularly still from 48.2% to 88.39% by weight, of at least one compound chosen from the elements of the group consisting of carbohydrates, inorganic salts and proteins,
   it being understood that the sum of the various constituents of this extract of fermented blueberries is equal to 100%.

According to a particular aspect, the carbohydrates present in the extract of blueberries are chosen from the elements of the group consisting of monosaccharides, such as glucose, xylose, arabinose, mannose, galactose or galacturonic acid; disaccharides, such as sucrose; polysaccharides, such as pectins, propectins or celluloses.

The extract of blueberries, preferably an extract of fermented blueberries, can be incorporated in an ingestible composition (IC). The extract of blueberries, preferably an extract of fermented blueberries, can be incorporated in an ingestible composition which does not comprise any other extract of fruits or vegetables.

The ingestible composition (IC) can also comprise at least one excipient which can be administered orally, said excipient being a pharmaceutically or nutritionally acceptable technological additive (TA) chosen from a diluting agent, a flow agent, a binding agent or a disintegrating agent.

The term “technological additive” denotes any chemical substance or any chemical composition, the technical function of which is to make possible and/or to facilitate the mixing of the various constituents of said composition (IC), to facilitate and/or to optimize the physical properties of said composition (IC), such as to facilitate and/or to optimize its flow, its stability and its incorporation in a subsequent pharmaceutical and/or nutritional formulation, and which are able to meet the conditions required by the regulations in force for the marketing of a pharmaceutical formulation and/or of a nutritional formulation.

The term “nutritionally acceptable technological additive” denotes a technological additive as defined above, the use of which meets the requirements of the regulations relating to food supplements in force in a country under consideration.

The ingestible composition (IC) can comprise at least one technological additive (TA) chosen from diluting agents, flow agents, binding agents or disintegrating agents.

The term “pharmaceutically acceptable technological additive” denotes a technological additive as defined above, the use of which meets the requirements of the pharmaceutical regulations in force in a country concerned. Typically, the diluting agent is chosen from at least one of the elements of the group consisting of lactose, sucrose, saccharose, glucose, maltodextrin, mannitol, sorbitol, xylitol, isomalt, calcium hydrogen phosphate, microcrystalline cellulose, starches and more particularly corn starches, wheat starches and potato starches, dicalcium phosphate, anhydrous dibasic calcium phosphate, sodium carbonate, calcium carbonate and magnesium carbonate, monoglycerides and/or diglycerides of fatty acids comprising from 8 to 24 carbon atoms.

Typically, the flow agent is chosen from at least one of the elements of the group consisting of magnesium stearate, talc, sodium stearyl fumarate, hydrogenated vegetable oils, anhydrous colloidal silica, sodium benzoate and silicon dioxide.

Typically, the binding agent is chosen from at least one of the elements of the group consisting of starches in the form of starch pastes, pregelatinized starches, hydroxypropyl methyl cellulose, methyl cellulose, sucrose syrups and acacia gum.

Typically, the disintegrating agent is chosen from at least one of the elements of the group consisting of starches, sodium starch glycolate, alginic acid, sodium alginate, croscarmellose sodium, crospovidone and polyvinylpyrrolidone.

According to a particular aspect, the ingestible composition (IC), which is provided in the form of a solid, takes the form of a tablet, of a hard gelatin capsule, of a soft capsule, of a powder, of a sugar-coated tablet or of a granule.

The ingestible composition (IC) comprises a concentration of extract of blueberries, preferably an extract of fermented blueberries, expressed in milligrams of extract of blueberries per milligram of ingestible composition (IC), of greater than or equal to 1 and less than or equal to 1000, more particularly of greater than or equal to 20 and less than or equal to 800, more particularly of greater than or equal to 50 and less than or equal to 500, more particularly still of greater than or equal to 80 and less than or equal to 250.

The extract of fermented blueberries is manufactured according to the process below and comprising the following stages:
the first stage of the process thus consists of a fermentation of the fruit of Vaccinium myrtillus (blueberry) in the presence of Saccharomyces cerevisiae,
second stage: the fermentation medium is subsequently filtered,
third stage: the filtered medium is concentrated,
fourth stage: the concentrated medium is taken up in ethanol so as to remove all the soluble compounds which are contained in the ethyl alcohol,
fifth stage: the extract is filtered,
sixth stage: the extract is dried,
seventh stage: the isolated solid obtained above is subsequently redissolved in water,
eighth stage (optional): the aqueous phase obtained above is subjected to atomization in order to obtain a solid which is subsequently packaged.

It should be noted that this process can be carried out without a prior fermentation stage, replaced by a stage of grinding the fruits: the corresponding extract then exhibits a different chemical composition.

The differences in chemical compositions between an extract of fermented and nonfermented blueberries are significant.

An extract of nonfermented blueberries contains predominantly anthocyanins corresponding to phenolic glycosides. When a fermentation stage is carried out, these anthocyanins undergo various consecutive and/or sequential chemical transformations, such as deglycosylation, reduction or polymerization reactions, to finally result in two distinct polymers: proanthocyanidins (flavanol homopolymers) and pseudoproanthocyanidins (flavanol polymers, the end of which is terminated by an anthocyanidin unit).

These reaction sequences and also the chemical structure of a flavanol are illustrated below.

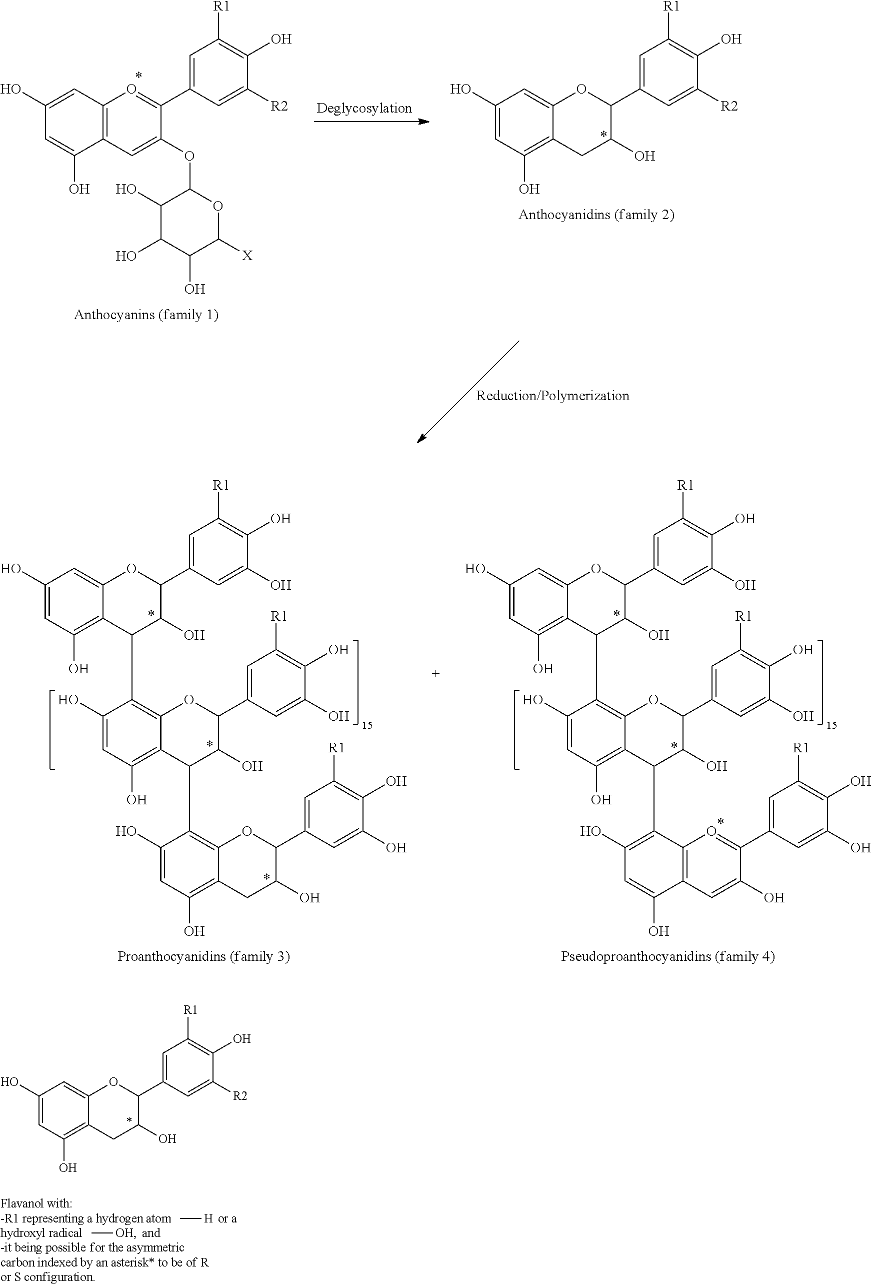

The ingestible composition (IC) comprising the extract of blueberries as the sole fruit extract, without other fruit or vegetable extracts, can be included in a food supplement.

According to a particular aspect of the present invention, said food supplement does not comprise another extract of fruits or vegetables.

Preferably, the food supplement comprises, per 100% of its weight, from 5% to 70% by weight, more particularly from 10% to 70% by weight and more particularly still from 25% to 70% by weight of said ingestible composition (IC).

The term “food supplement” is understood to mean a foodstuff, the purpose of which is to supplement the normal diet and which constitutes a concentrated source of nutrients or other substances having a nutritional or physiological effect alone or in combination. The food supplement makes it possible to prevent certain deficiencies or to respond to specific needs in the diet of an individual, in particular during physical exertion. This definition of “food supplement” is given in Article 2 of Decree No. 2006-20 352 of Mar. 26, 2006, of the French Republic concerning food supplements and in Directive 2002/46/EC of the European Parliament and of the Council of Jun. 10, 2002.

The food supplement is in solid or liquid form.

The food supplement additionally comprises at least one active principle chosen from bioactive lipids, water-soluble or water-dispersible trace element salts, vitamins which are water-soluble or fat-soluble, prebiotics, probiotics, milk protein concentrates and/or proteins, plant or animal enzymes, amino acids, peptides or sugars.

The food supplement can also be provided in any formulation form known to a person skilled in the art, such as in the form of a tablet, of a hard gelatin capsule, of a sugar-coated tablet, of a granule, of a soft capsule, of a syrup, of a powder, such as an immediate-release powder, of a delayed-release powder or of a powder for reconstituted drinks, of a liquid, of a stick or of a gel.

The solid forms used in the industries of food supplements are generally provided in the form of tablets, of hard gelatin capsules, of sugar-coated tablets or of granules which are made by agglomeration of solid particles comprising at least one nutritional ingredient.

These solid forms can be prepared by the implementation of many techniques known to a person skilled in the art, such as compression, pelletization, granulation, compacting or extrusion techniques.

When the food supplement is provided in the form of a powder, it is obtained by introduction of its various constituents into a mixer equipped with at least one mechanical stirring system, such as flat or impeller-type stirring blades, and the mixer is optionally a tumbler mixer, and the mixer is optionally equipped with a system of lump breaker type. This mixing operation is generally carried out at ambient temperature.

The food supplement can be provided in any form of food product known to a person skilled in the art, such as a drink, and more particularly an aqueous drink, a solution, a fruit juice, a flavored drink, an energy drink, an alcoholic drink, a coffee-based drink, a chocolate-based drink, a tea-based drink, a milk product, and more particularly milk, yoghurt, a milk desert, drinkable yoghurt, a cheese, an ice cream, a chocolate bar, a cereal product, and more particularly a cereal bar, a cookie, breakfast cereals, flours, bread-making products, a specialized nutrition product, more particularly an infant nutrition product, a nutrition product for preparing for physical exertion, a clinical nutrition product, a meal substitute, confectionery, more particularly chewing gums, candies, caramels, sugared almonds, humbugs, marshmallows, Turkish delight, nougats, fruit jellies, licorice.

Mention may be made, as bioactive lipids optionally present in the food supplement, of phytosterols, such as those extracted from vegetable oils, and more particularly extracts of sea buckthorn oil, of corn oil or of soybean oil; complexes of phytosterols, isolated from vegetable oils, such as cholestatin, composed of campesterol, of stigmasterol and of brassicasterol; phytostanols; carotenoids, which belong to the family of the terpenoids, extracted from algae, green plants, fungi or bacteria; polyunsaturated fatty acids of the omega-3 group, such as α-linolenic acid, eicosapentaenoic acid or docosahexanoic acid; polyunsaturated fatty acids of the omega-6 group, such as linoleic acid, γ-linolenic acid, eicosadienoic acid, dihomo-γ-linolenic acid, arachidonic acid, docosadienoic acid, docosatetraenoic acid or docosapentaenoic acid.

Mention may be made, as examples of water-soluble or water-dispersible trace element salts optionally present in the food supplement, of ferrous carbonate, ferrous chloride tetrahydrate, ferric chloride hexahydrate, ferrous citrate hexahydrate, ferrous fumarate, ferrous lactate tetrahydrate, ferrous sulfate monohydrate, ferrous sulfate heptahydrate, ferrous amino acid chelate hydrates, iron glycine chelate, calcium iodate hexahydrate, anhydrous calcium iodate, sodium iodide, potassium iodide, cobalt acetate tetrahydrate, basic cobalt carbonate monohydrate, cobalt carbonate hexahydrate, cobalt sulfate heptahydrate, cobalt sulfate monohydrate, cobalt nitrate hexahydrate, cupric acetate monohydrate, basic copper carbonate monohydrate, cupric chloride dihydrate, copper methionate, cupric sulfate pentahydrate, cuprous amino acid chelate hydrates, cuprous glycine chelate hydrate, copper chelate of hydroxy analog of methionine, manganous carbonate, manganous chloride tetrahydrate, manganese hydrogen phosphate trihydrate, manganous sulfate tetrahydrate, manganous sulfate monohydrate, manganese amino acid chelate hydrate, manganese glycine chelate hydrate, manganese chelate of hydroxy analog of methionine, ammonium molybdate, sodium molybdate, sodium selenite, sodium selenate, the organic form of selenium produced by Saccharomyces cerevisiae, selenomethionine (inactivated selenium-containing yeast) and the selenomethionine produced by Saccharomyces cerevisiae (inactivated selenium-containing yeast).

Mention may be made, as examples of water-soluble or fat-soluble vitamins optionally present in the food supplement, of vitamin A, more particularly in its form of retinol, of retinyl acetate, of retinyl palmitate or of β-carotene, vitamin D2, more particularly in its form of ergocalciferol or of 25-hydroxycalciferol, vitamin D3, more particularly in its form of cholecalciferol, vitamin K, more particularly in its form of phylloquinone (phytomenadione) or of menaquinone, vitamin B1, more particularly in its form of thiamine hydrochloride, of thiamine mononitrate, of thiamine monophosphate chloride or of thiamine pyrophosphate chloride, vitamin B2, more particularly in its form of riboflavin or of sodium riboflavin 5′-phosphate, vitamin B6, more particularly in its form of pyridoxine hydrochloride, of pyridoxine 5′-phosphate or of pyridoxal 5′-phosphate, vitamin B12, more particularly in its form of cyanocobalamin, of hydroxocobalamin, of 5′-deoxyadenosylcobalamin or of methylcobalamin, vitamin C, more particularly in its form of L-ascorbic acid, of sodium L-ascorbate, of calcium L-ascorbate, of potassium L-ascorbate, of calcium salts of 6-palmitoyl-L-ascorbic acid or of sodium ascorbyl monophosphate, pantothenic acid, more particularly in its form of calcium D-pantothenate, of sodium D-pantothenate, of dexpanthenol or of pantethine, vitamin PP, more particularly in its form of nicotinic acid, of niacin, of nicotinamide or of inositol hexanicotinate (inositol hexaniacinate), vitamin B9, more particularly in its form of folic acid, folates, more particularly in their form of pteroylmonoglutamic acid, of calcium L-methylfolate or of (6S)-5-methyltetrahydrofolic acid in the glucosamine salt form, vitamin H2, B7 or BW, more particularly in its form of biotin, choline, more particularly in its form of choline chloride, of choline dihydrogen citrate or of choline bitartrate, inositol, carnitine, more particularly in its form of L-carnitine or L-carnitine L-tartrate, or taurine.

Mention may be made, as examples of prebiotics optionally present in the food supplement, of inulin, trans-galacto-oligosaccharides, fructans and manno-oligosaccharides.

Mention may be made, as examples of probiotics optionally present in the food supplement, of various strains of Saccharomyces cerevisiae, of Bacillus cereus var. toyoi, of Bacillus subtilis, alone or in combination with Bacillus licheniformis, or also strains of Enteroccocus faecium. These strains of microorganisms are generally combined with a solid support, for example calcium carbonate, dextrose or sorbitol.

Mention may be made, as examples of milk protein concentrates and/or proteins optionally present in the food supplement, of milk proteins resulting from milk cracking, such as colostrum in the form of a lyophilized or atomized powder, whey in the form of a powder, of fractions which are purified or enriched in IgG, in lactoferrin or in lactoperoxidase.

Mention may be made, as examples of plant or animal enzymes optionally present in the food supplement, of Promutase, superoxide dismutase (SOD), 3-phytase, 6-phytase, endo-1,4-β-glucanases, endo-1,4-β-xylanases, or also other enzymes which improve or promote digestion.

Mention may be made, as examples of peptides optionally present in the food supplement, of avocado peptides, lupin peptides, quinoa peptides, maca peptides, fermented or unfermented soybean peptides, rice peptides, peptides present in Acacia macrostachya seed extract or peptides present in passionflower seed extracts.

Mention may be made, as examples of amino acids optionally present in the food supplement, of alanine, arginine, asparagine, aspartic acid, cysteine, glutamic acid, glutamine, glycine, histidine, isoleucine, leucine, lysine, methionine, phenylalanine, proline, hydroxyproline, pyrrolysine, selenocysteine, serine, threonine, tryptophan, tyrosine, valine, sarcosine or ornithine.

Mention may be made, as examples of sugars optionally present in the food supplement, of water-soluble polysaccharides, or sugars of lower molecular weight, such as oligosaccharides or mono- or disaccharides, such as glucose, lactose or dextrose.

Mention may be made, as examples of excipients optionally present in the food supplement which is a subject matter of the present invention, of flavor enhancers and flavoring agents.

Mention may be made, as examples of flavor enhancers optionally present in the food supplement, of glutamates, such as glutamic acid, monosodium glutamate, monopotassium glutamate, calcium diglutamate, ammonium glutamate or magnesium diglutamate, guanylates, such as guanylic acid (guanosine monophosphate), disodium guanylate, dipotassium guanylate or calcium guanylate, or inosinates, such as inosinic acid, disodium inosinate, dipotassium inosinate or calcium inosinate.

Mention may be made, as examples of sweeteners optionally present in the food supplement, of Stevia extracts or rebaudiosides.

The invention also relates to a method for improving the esthetic appearance of human skin, the improvement to said esthetic appearance of human skin comprising both the improvement in its coloration and in the uniformity of its complexion and also the decrease in its roughness, said method being characterized in that an effective amount of an extract of blueberries is orally administered to the person.

The method according to the invention can exhibit one or more of the following characteristics:
the effective amount of said extract of blueberries administered orally is at least 100 mg of said extract of blueberries once daily;
the effective amount of said extract of blueberries administered orally is at least 100 mg of said extract of fermented blueberries once daily;
the extract of fermented blueberries comprises, per 100% of its weight:
   from 20% to 50% by weight of at least one compound of formula (I),
   from 0.01% to 5% by weight of at least one compound of formula (II),
   from 1% to 5% by weight of at least one compound of formula (III),
   from 1% to 10% by weight of water,
   from 30% to 78.99% of at least one compound chosen from the group consisting of carbohydrates, inorganic salts and proteins,
it being understood that the sum of the various constituents of this extract of fermented blueberries is equal to 100%;
the extract of fermented blueberries comprises, per 100% of its weight:
   from 0.1% to 5% by weight of at least one compound of formula (I),
   from 0.01% to 1% by weight of at least one compound of formula (II),
   from 0.5% to 10% by weight of at least one compound of formula (III),
   from 10% to 50% by weight of at least one compound of formula (IV),
   from 1% to 10% by weight of water,
   from 24% to 88.39% by weight of at least one compound chosen from the elements of the group consisting of carbohydrates, inorganic salts and proteins,
it being understood that the sum of the various constituents of this extract of fermented blueberries is equal to 100%.

### EXAMPLES

An extract of fermented blueberries, obtained according to the process described above and sold under the brand name Extracyan™ by Ferlux (hereinafter composition EB1), is used. The analytical characteristics of the composition (EB1) are as follows:
water: 2.1%,
anthocyanins or compounds of formula (I): 0.8% (*)
anthocyanidins or compounds of formula (II): 0.03% (*)
proanthocyanidins or compounds of formula (III): 2.0% (*)
pseudoanthocyanidins or compounds of formula (IV): 16% (*)
carbohydrates, inorganic salts and proteins: 79.07% (*) percentage by weight obtained by the implementation of an analytical quantification method using a high-pressure liquid chromatograph (HPLC), and expressed per 100% of the extract.

The demonstration of the technical effects making it possible to solve the technical problem was carried out with an ingestible composition comprising the extract of fermented blueberries, denoted composition (C1) and described in table 1 below.

TABLE 1
Composition (C1)

| Component | Amount daily dose |
|---|---|
| Extract of fermented blueberries (EB1) | 100 mg |
| Maltodextrin | 334 mg |
| Mg stearate | 10 mg |
| Total net weight | 444 mg |
| Total weight (with hard gelatin capsule) | 539 mg |

A placebo composition (C0) will also be tested in comparison with the composition (C1); such a composition (C0) is described in table 2 below.

TABLE 2
Placebo composition (C0)

| Component | Amount daily dose |
|---|---|
| Extract of fermented blueberries (EB1) | — |
| Maltodextrin | 434 mg |
| Mg stearate | 10 mg |
| Total net weight | 444 mg |
| Total weight (with hard gelatin capsule) | 539 mg |

As mentioned above, a healthy complexion of the skin can be defined by several parameters, including the coloration of the skin (good look effect), the uniformity of the complexion and a smooth skin grain.

### The Color of the Skin

The color of the skin can be analyzed using the CIELAB or L*a*b* system. The parameters of the L*a*b* system are:
the lightness L*, which takes values between 0 (black) and 100 (reference white),
the parameter a*, representing the value on a green→red axis,
the parameter b*, representing the value on a blue→yellow axis, and
the ITA° (Individual Typology Angle) parameter which results from the parameters L* and b* and represents a dark to light complexion.

A “good look” effect and an improvement in the coloration of the complexion and an improvement in the complexion of the skin will tend to be reflected by:
an increase in the parameters a* and b* and
a decrease in the ITA° associated with a darker complexion.

### Uniformity of the Complexion

The skin is a richly vascularized tissue. The capillaries and arterioles which make up this vascular system at the level of the epidermis find their blood flow subjected to modifications in the event of a change in temperature, of stress, such as pudic erythema, or relaxation/vasodilation which are responsible for rosacea, for example.

This can be reflected by the appearance of diffuse blotches or transient or permanent red patches due to vasodilation of the capillaries.

The effect desired for a more uniform complexion is the improvement in the vasoconstrictive properties of a substance or of a composition, which contribute to reducing vasodilation defects, and thus imperfections, for a more uniform complexion. These properties can be studied by measurement of blood perfusion by doppler laser imaging and by evaluation of the uniformity of this perfusion by a dermatologist.

The uniformity of the complexion can also be directly evaluated by a dermatologist.

The PeriCam PSI™ system, sold by Perimed, is a blood perfusion imaging system based on laser speckle contrast analysis technology. This system uses a near infrared (785 nanometers) laser for the blood perfusion measurements. The beam is distributed over the measurement zone by a diffuser, creating a speckle pattern (marks).

This speckle pattern is recorded with a CCD camera and the blood perfusion is calculated by analyzing the variations in this speckle pattern.

The dermatologist evaluation is carried out on photos using scoring scales (cf. tables 3 and 4).

TABLE 3
Evaluation of the uniformity of the blood perfusion.

| Score | Description |
|---|---|
| 1 | Very nonuniform blood perfusion |
| | Presence of red areas covering almost the entire face |
| 2 | Nonuniform blood perfusion |
| | Presence of many red areas covering the face |
| 3 | Fairly uniform blood perfusion |
| | Predominance of green/yellow areas and a few small |
| | red areas on the face |
| 4 | Uniform blood perfusion |
| | Green/yellow areas over the entire face and no red area |
| | visible |

TABLE 4
Evaluation of the uniformity of the complexion.

| Score | Description |
|---|---|
| 1 | Nonuniform complexion |
| | Presence of discolorations/dark spots over the entire |
| | face |
| 2 | Nonuniform complexion |
| | Presence of discolorations/dark spots on certain parts |
| | of the face |
| 3 | Fairly uniform complexion |
| 4 | Uniform complexion |

### Skin Grain

A smoother skin grain also contributes to an improvement in skin complexion by its texture and the decrease in its roughness.

The roughness of the skin can be evaluated by a dermatologist, again on photos using a scale of scoring of the wrinkles (cf. table 5).

TABLE 5
Evaluation of the roughness of the skin.

| Score | Description |
|---|---|
| 0 | No visible wrinkle, continuous skin line |
| 0.5 | Very superficial but visible wrinkles |
| 1 | Visible wrinkles and slight hollow |
| 1.5 | Visible wrinkles and obvious hollow |
| 2 | Wrinkles clearly visible |
| 2.5 | Visible and significant wrinkles |

### Forms of the Study on the Extract

The technical effect of the composition (C1) was demonstrated in a pilot, randomized, double-blind, parallel-group clinical study.

It made it possible to test the effect of the daily consumption (1 hard gelatin capsule per day) of 100 mg of the extract (EB1) equivalent to 539 mg of the composition (C1) versus the placebo composition (C0) over a period of 84 days (12 weeks).

Women aged between 18 and 65 years were included in the study with the following criteria: Caucasian ethnicity, phototype I to III with a moderately to very dull skin complexion. The measurements were carried out on day 0 (D0) and 84 days after consumption of the compositions (C1) or (C0) (D84).

### a) Results Obtained for the Color of the Skin

The change in the color of the skin was carried out by virtue of the monitoring of the parameters a*, b* and ITA°, which are obtained with a CM-700d spectrophotometer-colorimeter (sold by Konica Minolta) according to the CIELAB standard method defined by the International Commission on Illumination (C.I.E.)

A significant increase in the parameter a* was observed in the composition (C1) group after 84 days of consumption: from 12.01±0.28 a.u. (arbitrary unit) at D0 to 12.62±0.29 a.u. at D84, p=0.012, whereas there was no significant change in the placebo composition (C0) group: from 12.05±0.33 a.u. at D0 to 12.07±0.34 a.u. at D84.

Also, a significant increase in the parameter b* was observed in the composition (C1) group after 84 days of consumption: from 16.68±0.33 a.u. at DO to 17.29±0.42 a.u. at D84 (p=0.028), whereas there was no significant change in the placebo composition (C0) group: from 16.62±0.37 a.u. at D0 to 16.49±0.47 a.u. at D84. In addition, there was a statistically significant difference between the composition (C1) group (4.0±1.7%) and the placebo composition (C0) group (−0.9±1.7%, p=0.047).

A significant decrease in the parameter ITA° was observed in the composition (C1) group after 84 days of consumption: from 40.39±1.13 a.u. at D0 to 38.39±1.16 a.u. at D84 (p=0.003), whereas there was no significant change in the placebo composition (C0) group: from 40.52±1.10 a.u. at D0 to 40.24±1.31 a.u. at D84.

### b) Uniformity of the Complexion

The blood perfusion was evaluated by virtue of a doppler laser perfusion imaging system (PeriCam PSI System, sold by Perimed).

A significant decrease in the blood perfusion was observed in the composition (C1) group after 84 days of consumption: from 139.8±5.5 a.u. at D0 to 118.0±5.3 a.u. at D84 (p<0.001), whereas there was no significant change in the placebo composition (C0) group: from 138.1±6.8 a.u. at D0 to 127.0±6.6 a.u. at D84.

The uniformity of the blood perfusion and the uniformity of the complexion were evaluated by a dermatologist on photographs of the faces of the subjects taken at D0 and D84, according to the scoring method described in the preceding tables 4 and 5.

An improvement in the uniformity of the blood perfusion was noted in 62.1% of the subjects in the composition (C1) group, whereas it was 30% in the placebo composition (C0) group.

An improvement in the uniformity of the complexion of the skin was noted in 69% of the subjects in the composition (C1) group, whereas it was 46.7% in the placebo composition (C0) group.

An improvement in the softness of the skin was noted in 80% of the subjects in the composition (C1) group, whereas it was 30% in the placebo group.

### c) Grain and Roughness of the Skin

The grain and the roughness of the skin were evaluated by a dermatologist on photographs of the faces of the subjects taken at D0 and D84, according to the scoring method described in the preceding table 6.

A decrease in the roughness of the skin and thus a smoother skin grain were noted in 80% of the subjects in the composition (C1) group. This decrease was only 30% in the placebo composition (C0) group.

### d) Conclusion

The composition (C1) has demonstrated in the clinical study its ability to improve, in a combined manner, the coloration of the skin, the uniformity of the complexion and the skin grain.

Furthermore, the antioxidant properties of proanthocyanidins and anthocyanins are already known. There was thus a wish to evaluate the change in the antioxidant activity at the level of the skin in the clinical study.

Samples were taken from the skin with Corneofix® adhesive patches (Courage+Khazaka electronic GmbH). Patches numbers 2 and 3 were used for the FRAP (Ferric Reducing Antioxidant Parameter). This is a direct measurement of the reducing power of the sample which makes it possible to evaluate the ability to withstand oxidative damage. The reduction at acidic pH of Fe³⁺ ions to give Fe²⁺ ions is characterized by the appearance of the color blue, which is monitored by colorimetry at 595 nanometers.

A significant increase in the antioxidant activity was observed in the composition (C1) group after 84 days of consumption: from 69.6±3.8 μM Fe²⁺ equ. at D0 to 85.7±4.9 μM Fe²⁺ equ. at D84 (p<0.001), whereas there was no significant change in the placebo composition (C0) group: from 70.9±3.1 μM Fe²⁺ equ. at D0 to 76±4.4 μM Fe²⁺ equ. at D84. In addition, there was a statistically significant difference between the composition (C1) group (26.5±3.7%) and the placebo composition (C0) group (7.1±3.8%, p<0.001).

The benefit perceived by the subjects of the study was also evaluated by virtue of a self-assessment questionnaire.

At the end of the study (D84), the subjects gave their opinion on the ingredients in the test. 83% of the subjects in the composition (C1) group considered that the complexion was improved, whereas they were 57% in the placebo composition (C0) group. 69% of the subjects in the composition (C1) group saw a reduction in their imperfections, whereas they were 33% in the placebo composition (C0) group. 82% considered that they had fewer red patches in the composition (C1) group, whereas they were 40% in the placebo composition (C0) group. 90% perceived their skin to be more hydrated in the composition (C1) group, whereas they were 57% in the placebo composition (C0) group. Finally, 69% of the subjects in the composition (C1) group considered that the skin grain was finer, whereas they were 37% in the placebo composition (C0) group.

### Formulation Examples

TABLE 6
Formula for hard gelatin capsules.

| Component | Amount (mg) | |
|---|---|---|
| Extract of fermented blueberries (EB1) | 100 | mg |
| Maltodextrin | 330.55 | mg |
| Magnesium stearate | 10 | mg |
| Silicon dioxide | 4.45 | mg |

TABLE 7
Formula for sticks.

| Component | Amount (mg) |
|---|---|
| Extract of fermented blueberries (EB1) | 100 mg |
| Maltodextrin | 1400 mg |

1. The use of an extract of blueberries for improving the esthetic appearance of human skin, the improvement in said esthetic appearance of human skin comprising both that in its coloration, in the uniformity of its complexion and also the decrease in its roughness.

2. The use as claimed in claim 1, wherein the extract of blueberries is an extract of fermented blueberries.

3. The use as claimed in claim 1, wherein the extract of fermented blueberries comprises the following compounds:
a) at least one compound of formula (I): 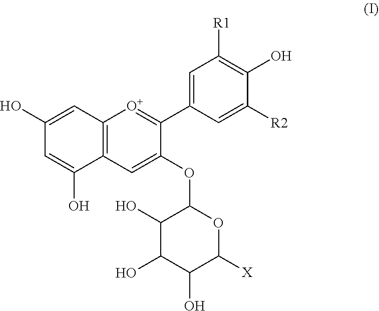
with R1 and R2 identical or different and representing a hydrogen atom —H, the hydroxyl radical —OH or the methoxyl radical —OCH₃,
and
with X═—H (L-arabinose) or —CH₂—OH (D-glucose, D-galactose);
b) at least one compound of formula (II): 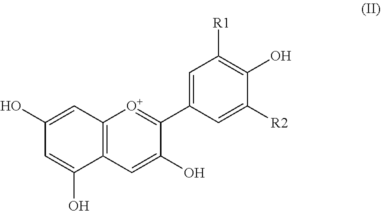
with R1 and R2 identical or different and representing a hydrogen atom —H, the hydroxyl radical —OH or the methoxyl radical —OCH₃; and
c) at least one compound of formula (III): 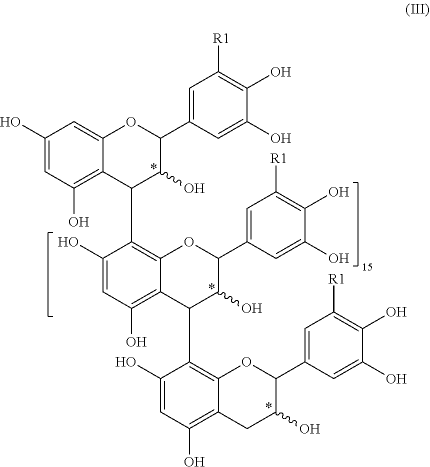
with, independently for each repeat and constituent unit of the compound of formula (III):
R1 representing a hydrogen atom —H or the hydroxyl radical —OH, and
it being possible for the asymmetric carbons indexed by asterisks * in the formula (III) to be of R or S configuration.

4. The use as claimed in claim 3, wherein the extract of fermented blueberries comprises, per 100% of its weight:
from 20% to 50% by weight of at least one compound of formula (I),
from 0.01% to 5% by weight of at least one compound of formula (II),
from 1% to 5% by weight of at least one compound of formula (III),
from 1% to 10% by weight of water,
from 30% to 78.99% of at least one compound chosen from the group consisting of carbohydrates, inorganic salts and proteins,
it being understood that the sum of the various constituents of this extract of fermented blueberries is equal to 100%.

5. The use as claimed in claim 1, wherein the extract of fermented blueberries comprises at least one compound of formula (IV): 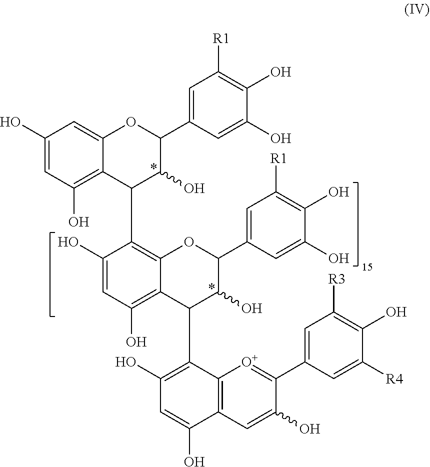
with, independently for each repeat and constituent unit of the compound of formula (IV):
R1 representing a hydrogen atom —H or the hydroxyl radical —OH, and
it being possible for the asymmetric carbons indexed by asterisks * in the formula (IV) to be of R or S configuration,
and
with R3 and R4 identical or different and representing a hydrogen atom —H, the hydroxyl radical —OH or the methoxyl radical —OCH₃;

6. The use as claimed in claim 5, wherein the extract of fermented blueberries comprises, per 100% of its weight:
from 0.1% to 5% by weight of at least one compound of formula (I),
from 0.01% to 1% by weight of at least one compound of formula (II),
from 0.5% to 10% by weight of at least one compound of formula (III),
from 10% to 50% by weight of at least one compound of formula (IV),
from 1% to 10% by weight of water,
from 24% to 88.39% by weight of at least one compound chosen from the elements of the group consisting of carbohydrates, inorganic salts and proteins,
it being understood that the sum of the various constituents of this extract of fermented blueberries is equal to 100%.

7. A method for improving the esthetic appearance of human skin, the improvement to said esthetic appearance of human skin comprising both that in its coloration and the uniformity of its complexion and also the decrease in its roughness, said method being wherein an effective amount of an extract of blueberries is orally administered to the person.

8. The method as claimed in claim 7, wherein the effective amount of said extract of blueberries administered orally is at least 100 mg of said extract of blueberries once daily.

9. The method as claimed in claim 7, wherein the extract of fermented blueberries comprises, per 100% of its weight:
from 20% to 50% by weight of at least one compound of formula (I),
from 0.01% to 5% by weight of at least one compound of formula (II),
from 1% to 5% by weight of at least one compound of formula (III),
from 1% to 10% by weight of water,
from 30% to 78.99% of at least one compound chosen from the group consisting of carbohydrates, inorganic salts and proteins,
it being understood that the sum of the various constituents of this extract of fermented blueberries is equal to 100%.

10. The method as claimed in claim 7, wherein the extract of fermented blueberries comprises, per 100% of its weight:
from 0.1% to 5% by weight of at least one compound of formula (I),
from 0.01% to 1% by weight of at least one compound of formula (II),
from 0.5% to 10% by weight of at least one compound of formula (III),
from 10% to 50% by weight of at least one compound of formula (IV),
from 1% to 10% by weight of water,
from 24% to 88.39% by weight of at least one compound chosen from the elements of the group consisting of carbohydrates, inorganic salts and proteins,
it being understood that the sum of the various constituents of this extract of fermented blueberries is equal to 100%.
